(19) United States
(12) Patent Application Publication
(10) Pub. No.: US 2024/0060983 A1
(43) Pub. Date: Feb. 22, 2024

(54) METHODS FOR ASSAYING TARGET PROTEINS ON EXTRACELLULAR VESICLES IN PLASMA

(71) Applicant: Verily Life Sciences LLC, South San Francisco, CA (US)

(72) Inventors: Cory Batenchuk, South San Francisco, CA (US); Jia Lin, Redwood City, CA (US); Kimary Kulig, San Francisco, CA (US); Adrian Lobito, San Francisco, CA (US); Daniel Liu, San Mateo, CA (US); Wilman Luk, South San Francisco, CA (US); James Sullivan, San Francisco, CA (US)

(73) Assignee: Verily Life Sciences LLC, South San Francisco, CA (US)

(21) Appl. No.: 18/268,925

(22) PCT Filed: Dec. 21, 2021
(86) PCT No.: PCT/US2021/064681

§ 371 Date: Jun. 21, 2023

Related U.S. Application Data
(60) Provisional application No. 63/199,379, filed on Dec. 22, 2020.

(51) Int. Cl. G01N 33/574; G01N 33/542; C07K 16/28

(52) U.S. Cl. CPC G01N 33/57488; G01N 33/542; C07K 16/2827; C07K 16/2818

(57) ABSTRACT

Provided herein are compositions and methods for assaying target proteins on extracellular vesicles in plasma. In some methods, the responsiveness of a subject to immunotherapy is determined.

### PRIOR RELATED APPLICATION

This application claims the benefit of U.S. Provisional Application No. 63/199,379 filed on Dec. 22, 2020, which is hereby incorporated by reference in its entirety.

### BACKGROUND

Immunotherapy has emerged as a promising treatment for diseases such as cancer. For example, cancer immunotherapy utilizing blockade of the PD-1/PD-L1 checkpoint has been approved by the Food and Drug Administration (FDA) for the treatment of several types of cancers. However, these therapies are ineffective in a significant percentage of patients, thus limiting their usefulness in a wide range of cancers and patients.

### SUMMARY

Programmed cell death protein ligand 1 (PD-L1) testing is used to predict the usefulness of anti-Programmed cell death protein 1 (PD-1) therapy in a patient, but the predictive value of current PD-L1 testing is poor. Additionally, challenges in acquisition of biopsies, for example, may result in exclusion of a patient as a candidate for anti-PDL1. The present method provides an novel method of screening for responsivity to an immunotherapy by determining the level of an extracellular vesicular (EV) protein expressed on cancer cells, for example, extracellular vesicular PD-L1 (EV PD-L1) in plasma to predict responsivity to PD-L1 in a subject. Thus, provided herein are methods for detecting, in a subject with cancer, the level of a target protein on the surface of extracellular vesicles (EVs). The method comprises detecting in a subject with cancer the level of a first target protein expressed on the surface of extracellular vesicles (EVs), by (a) contacting a plasma sample from the subject with (i) a first binding agent that specifically binds to the first target protein expressed on the surface of EVs, wherein the first binding agent is conjugated to a first member of a proximity pair; and (ii) a second binding agent that specifically binds to a second target protein, wherein the second target protein is selectively expressed on the surface of EVs and wherein the second binding agent is conjugated to a second member of a proximity pair; and (b) detecting the level of the first target protein expressed on the surface of the EVs by detecting proximity of the first member of the proximity pair and the second member of the proximity pair, wherein the proximity occurs upon binding of the first binding agent to the first target protein and binding of the second binding agent to the second target protein on the same EV.

In some methods, the proximity pair is a donor-acceptor pair and detecting proximity comprises detecting an interaction between a donor and an acceptor or a relay of chemical signal from the donor to the acceptor. When the first member of the donor-acceptor pair is an acceptor molecule, the second member of the donor-acceptor pair is a donor molecule. When the first member of the donor-acceptor pair is a donor molecule, the second member of the donor-acceptor pair is an acceptor molecule.

The first target protein is optionally selected from the group consisting of programmed cell death protein 1 (PD-1) (for example, the human PD-1 amino acid sequence set forth under UniProt. No. Q15116, or a fragment thereof), programmed cell death protein ligand 1 (PD-L1) (for example, the human PD-L1 amino acid sequence set forth under UniProt. No. Q9NZQ7, or a fragment thereof), programmed cell death protein ligand 2 (PD-L2) (for example, the human PD-L2 amino acid sequence set forth under UniProt. No. Q9BQ51, or a fragment thereof), cytotoxic T-lymphocyte-associated protein 4 (CTLA4) (for example, the human CTLA4 amino acid sequence set forth under UniProt. No. P16410, or a fragment thereof), Lymphocyte-Activation Gene 2 (LAG2) (for example, the human LAG2 amino acid sequence set forth under UniProt. No. P22749, or a fragment thereof), transforming growth factor (31 (TGF-β1) (for example, the human TGF-β1 amino acid sequence set forth under UniProt. No. Q9H2G4, or a fragment thereof), NKG2D (for example, the human NKG2D amino acid sequence set forth under UniProt. No. P26718, or a fragment thereof), T-cell immunoglobulin and mucin domain-3 (TIM-3) (for example, the human TIM-3 amino acid sequence set forth under UniProt. No. Q8TDQ0, or a fragment thereof), Lymphocyte-Activation Gene 3 (LAG-3) (for example, the human LAG-3 amino acid sequence set forth under UniProt. No. P18627, or a fragment thereof), and V-domain Ig suppressor of T cell activation (VISTA) (for example, the human VISTA amino acid sequence set forth under UniProt. No. Q9H7M9, or a fragment thereof). The second target protein is optionally selected from the group consisting of CD9 (for example, the human CD9 amino acid sequence set forth under UniProt. No. P21926, or a fragment thereof), CD63 (for example, the human CD63 amino acid sequence set forth under UniProt. No. P08962, or a fragment thereof), or CD81 (for example, the human CD81 amino acid sequence set forth under UniProt. No. P60033, or a fragment thereof). By way of example, the first target protein can be PD-L1, and the second target protein can be CD9, CD63 or CD81.

The first and second binding agents are optionally conjugated to the first and second members, respectively, of the proximity pair via a covalent or a biotin-streptavidin linkage. When the biotin-streptavidin linkage is utilized, the plasma sample can be contacted with a biotinylated first or second binding agent that specifically binds the first or second target protein, respectively, then subsequently contacted with a streptavidin-conjugated moiety comprising first or second member of the proximity pair. For example, a streptavidin donor or acceptor molecule could bind to a biotinylated first or second binding agent

In one implementation, the proximity pair are donor and acceptor molecules and proximity is determined by an AlphaLISA ° assay. Other possible implementations of the proximity pair include donor and acceptor molecules in a homogeneous time-resolved fluorescence (HTRF) assay and oligonucleotide strands in proximity-dependent ligation or proximity-extension assays.

Optionally, the EVs need not be isolated from the plasma sample.

The methods described herein can further comprise determining the responsiveness of the subject to an immunotherapy that specifically binds a cognate or engineered ligand or receptor of the first target protein, wherein an increase in the level of the first target protein as compared to a control indicates the subject is nonresponsive to the immunotherapy, and a decrease in the level of the first target protein as compared to a control indicates the subject is responsive to the immunotherapy. The increase or decrease in the level of the first target protein can be an increase or a decrease of at least about 10%, 20%, 30%, 40%, 50% or greater.

Optionally, the methods further comprise administering the immunotherapy to the subject that is deemed to be responsive to the immunotherapy. For example, the immunotherapy can be an anti-PD-1 therapy or an anti PD-L1 therapy, wherein a decrease in the level of EV PD-L1 as compared to a control indicates the subject is responsive to the anti-PD-1 therapy or the anti-PD-L1 therapy. In some methods, the anti-PD-1 therapy is a PD-1 or a PD-L1 inhibitor. In some methods, the inhibitor is an antibody. In some methods, the antibody is selected from the group consisting of pembrolizumab, nivolumab, atezolizumab, avelumab and durvalumab.

The methods described herein can be used in patients with various types of cancer. The cancer can be melanoma, non-small cell lung cancer, small cell lung carcinoma, squamous cell lung cancer, head and neck squamous cell carcinoma, renal cell carcinoma, urothelial carcinoma, breast cancer, Merkel cell carcinoma, hepatocellular carcinoma, esophageal carcinoma, gastric cancer, Hodgkin's lymphoma, cervical cancer, and endometrial cancer.

### DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is a schematic of an exemplary EV PD-L1 assay described herein.

FIG. 2 is a schematic showing that EVs (1) expressing a biomarker of interest ((2) PD-L1 protein), are bound to a biomarker recognition element ((3) high-affinity PD-1 molecule) that is linked to an AlphaLISA® Donor Bead (4). An AlphaLISA® Acceptor Bead (5) linked to a vesicle-specific antibody (6) binds to its cognate vesicle specific surface protein ((7) CD9 protein). Excitation by 680 nm light (8) causes the Donor Bead to emit single oxygen molecules (9) that travel in solution to activate the Acceptor Bead which then emits a sharp peak of light at 615 nm (10). This light emission is detected by an Alpha-enabled reader.

FIG. 3 shows that the EV PD-L1 assay detected vesicle-bound, but not free/soluble PD-L1. The assay does not register signal above background when increasing titrations of soluble PD-L1 are added to the assay (FIG. 3, left panel). Cell line-derived EVs co-expressing surface CD9 and PD-L1 spiked into an EV PD-L1 assay elicit signal in a vesicle/particle concentration dependent manner (FIG. 3, right panel).

FIG. 4 is the study design for assessment of the diagnostic use of an EV PD-L1 assay measuring EV PD-L1 and total PD-L1 (using a commercial ELISA kit) from the blood of non-small cell lung cancer patients prior to anti-PD-1/L1 therapy.

FIG. 5 shows that EV PD-L1 (y-axis) and total PD-L1 levels (x-axis) in pretreatment plasma samples are not correlated (Spearman correlation, r).

FIG. 6 shows that EV PD-L1 levels are significantly different (t-test, p-value<0.001) in pretreatment plasma from patients that exhibit tumor shrinkage or stable disease (I/O responders), as compared to patients whose tumors continue to grow during treatment (I/O non-responders) based on a 6 month RECIST data (left panel). However no significant difference in total PD-L1 was observed in I/O-responders and I/O non-responders pretreatment plasma samples. These data show elevated EV PD-L1 levels before therapy is associated with treatment response outcomes (right panel).

FIG. 7 shows the results of receiver operating characteristic (ROC) curve analysis which compared the accuracy of EV PD-L1 and total plasma PD-L1 assays in discriminating I/O-responder and I/O non-responder patients via analysis of pretreatment plasma samples. Area under the curve (AUC) calculations revealed the superior accuracy of EV PD-L1 vs total plasma PD-L1 in classifying treatment response outcomes.

### DETAILED DESCRIPTION

The following description recites various aspects of the compositions and methods described herein. No particular embodiment is intended to define the scope of the compositions and methods. Rather, provided are non-limiting examples of various compositions and methods that are at least included within the scope of the disclosed compositions and methods. The description is to be read from the perspective of one of ordinary skill in the art; therefore, information well known to the skilled artisan is not necessarily included.

Immunotherapy, for example, in the form of immune checkpoint inhibition, is often used as a first- or second-line treatment for several cancer types (e.g. non-small cell lung cancer, metastatic melanoma, and bladder cancer). Compared to chemotherapy, immunotherapy can effect longer periods of remission when used as a stand-alone agent or when combined with existing chemotherapy regimens. While having an important impact on patient outcome, immunotherapy is not always effective. For example, the response rate among non-small cell lung cancer patients treated with an anti-PD-1 agent is about 50%. For this reason, immunohistochemical measurement of PD-L1 in a tissue biopsy is approved as a diagnostic for various anti-PD-1 agents. Currently, PD-L1 testing is required as a companion diagnostic for the safe and effective use of anti-PD-1 therapies in breast, bladder, gastric/gastro-esophageal junction and non-small cell lung cancer (NSCLC). However, the predictive value of tissue PD-L1 is poor. Furthermore, as many as 40% of lung cancer patients are unable to provide a viable biopsy for PD-L1, as diagnosed with fine-needle aspirates or other cytology based methods. When PD-L1 companion diagnostic testing is required for the safe and effective use of the anti-PD-L1 agent, these patients are considered ineligible for immunotherapy.

EVs are secreted by all cell types, and cancer cells are especially prolific secretors. These cancer-derived EVs enter circulation and can display the same molecules that are found on the cancer cells (e.g., PD-L1), providing a readout of tissue PD-L1. Additionally, it has been reported that EVs displaying PD-L1 can engage the cognate receptor, PD-1, on immune cells to down-regulate immune activity, much like cancer cells do. It is possible that EV PD-L1 hinders T-cell immunity in a way that negates the effect of immunotherapy. Therefore, methods for accurately predicting a patient's response to an immunotherapeutic are necessary. As shown herein, measuring the level of an EV protein expressed on cancer cells, for example, EV PD-L1 in plasma, can be used to predict an immunotherapeutic response in a subject.

### Detection Methods

Provided herein is a method for detecting, in a subject with cancer, the level of a first target protein on the surface of EVs comprising (a) contacting a plasma sample from the subject with (i) a first binding agent that specifically binds to the first target protein expressed on the surface of EVs, wherein the first binding agent is conjugated to a first member of a proximity pair; and (ii) a second binding agent that specifically binds to a second target protein, wherein the second target protein is selectively expressed on the surface of EVs and wherein the second binding agent is conjugated to a second member of a proximity pair; and (b) detecting the level of the first target protein expressed on the surface of the EVs by detecting proximity of the first member of the proximity pair and the second member of the proximity pair, wherein the proximity occurs upon binding of the first binding agent to the first target protein and binding of the second binding agent to the second target protein on the same EV.

The contacting of the plasma sample with a first binding agent and a second binding agent can be performed separately but simultaneously or sequentially (e.g., the first binding agent before the second binding agent or vice versa). The first and second members of the proximity pair, for example, a donor and acceptor pair, are used such that one member of the proximity pair is conjugated to the first binding agent and the other member of the proximity pair is conjugated to the second binding agent, so as to allow proximity-dependent signal generation to result from the two binding agents binding to molecules on the same EV. By way of example, the proximity pair can be a donor-acceptor pair, wherein first and second members of a donor acceptor pair are used such that one member of the donor-acceptor pair is conjugated to a binding agent (e.g., the first binding agent) and the other member of the donor-acceptor pair is conjugated to the other binding agent (e.g., the second binding agent), so as to allow interaction of the first and second members of the donor-acceptor pair when the first and second binding agents are bound to first and second target EV proteins. Optionally, the proximity is determined using an AlphaLISA assay.

EVs are membrane-bound vesicles that are naturally released in vivo. As used herein, the term extracellular vesicle (EV) or exosome refers to a cell-derived vesicle, i.e., a lipid bilayer delimited particle, comprising a membrane that encloses an internal space (lumen). Generally EVs range in diameter from 20 nm to 1000 nm. EVs include, but are not limited to, vesicles derived from cells, vesiculated organelles, and vesicles produced by living cells (e.g., by direct plasma membrane budding or fusion of the late endosome with the plasma membrane).

In the methods provided herein, the first target protein is a protein expressed on EVs released or derived from cancer cells. The first target protein is not necessarily EV-specific, but is optionally a cancer-specific or tumor-specific protein. As used herein, EV-specific refers to expression limited to or substantially limited to expression on the surface of an EV. Cancer-specific or tumor-specific expression, as used herein, refers to expression that is limited to or substantially limited to expression by or on cancer cells. By way of example, the first target protein is selected from the group consisting of programmed cell death protein 1 (PD-1), programmed cell death protein ligand 1 (PD-L1), programmed cell death protein ligand 2 (PD-L2), cytotoxic T-lymphocyte-associated protein 4 (CTLA4), Lymphocyte-Activation Gene 2 (LAG2), transforming growth factor (31 (TGF-β1), NKG2D, T-cell immunoglobulin and mucin domain-3 (TIM-3), Lymphocyte-Activation Gene 3 (LAG-3), and V-domain Ig suppressor of T cell activation (VISTA).

In the methods provided herein, a second target protein is a protein that is specifically expressed (or selectively expressed) on the surface of EVs (i.e., EV-specific), and is different from the first target protein. As used herein, EV-specific expression refers to expression that is substantially limited to expression on EVs. In some methods, the second target protein is selected from the group consisting of CD9, CD63, CD8, an MHC class I protein, an MHC class II protein and an integrin. Examples of integrins include, but are not limited to, α6β4, αVβ5, α5β1, αVβ3, αVβ5, αVβ6, αVβ8, α4β7, αLβ2 and α3β1. To acquire the plasma sample, a blood sample can be drawn from a subject, followed by separation of a plasma sample from the blood sample. Methods for separating plasma from whole blood, including mechanical methods, such as sedimentation and centrifugation, are known to those of skill in the art. See, for example, Mukherjee et al., “Plasma separation from blood: the ‘lab on a chip’ approach,” Crit. Rev. Biomed Eng. 37(6): 517-29 (2009); and Tripathi et al., “Microdevice for plasma separation from whole human blood using bio-physical and geometrical effects,” Scientific Reports 6, Article Number 26749 (2016)). Although an EV isolation step can be used, EVs are not isolated from the plasma sample, allowing direct measurement of the level of the first target protein on EVs in the plasma sample.

Optionally, the proximity pair comprises a first member of a donor-acceptor pair and a second member of a donor-acceptor pair. The first member of the donor-acceptor pair is optionally an acceptor molecule and the second member of the donor-acceptor pair is a donor molecule. Alternatively, the first member of the donor-acceptor pair is optionally a donor molecule and the second member of the donor-acceptor pair is an acceptor molecule. In some methods, the first binding agent is conjugated to a donor molecule and the second binding agent is conjugated to an acceptor molecule. In some methods, the first binding protein is conjugated to an acceptor molecule and the second binding protein is conjugated to a donor molecule. In some methods, the first binding agent is conjugated to the first member of the donor-acceptor pair and/or the second binding agent is conjugated to the second member of the donor-acceptor pair. Methods to link the binding agent to the donor or acceptor molecule include but are not limited to, streptavidin-biotin linkageFLAG-anti-FLAG antibody linkage, Digoxin-anti-Dig antibody linkage, DNP-anti-DNP antibody linkage, physical absorption, and covalent coupling via amine or thiolate chemistries.

As used herein; a proximity pair is a pair of molecules, for example a first member and a second member of a proximity pair, that can be detected on the same vesicles, when the first member and the second member of the proximity pair are near or juxtaposed to each other. In some methods, the members of the proximity pair are within about 200 nm, 150 nm, 100 nm, 50 nm or less of each other. Optionally, the proximity pair comprises a first binding agent (e.g., a first antibody) that specifically binds a first target protein and a second binding agent (e.g., a second antibody) that specifically binds a second target protein, wherein, the first antibody and the second antibody are detected using proximity ligation assay, thus detecting the presence of the first and second target protein on the same vesicle. See, for example, Alam “Proximity Ligation Assay (PLA) Curr. Protoc. Immun. 123(1): e58 (2018); Greenwood et al. “Proximity assays for sensitive quantification of proteins,” Biomol. Detect. Quantif. 4: 10-16 (2015); Jalili et al. “Streamlined circular proximity ligation assay provides high stringency and compatibility with low-affinity antibodies,” PNAS 115(5): E925-E933 (2018).

Optionally, the first member of the proximity pair is a first unique oligonucleotide and the second member of the proximity pair is a second unique oligonucleotide, wherein the first and second oligonucleotides hybridize to each other when the oligonucleotides are in proximity to each other. The hybridized product can be extended by DNA polymerase and subsequently detected and quantified by quantitative real-time PCR. See, for example, Thorsen et al. “Detection of serological biomarkers by proximity extension assay for detection of colorectal neoplasias in symptomatic individuals,” Journal of Translational Medicine 11, Article 253 (2013).

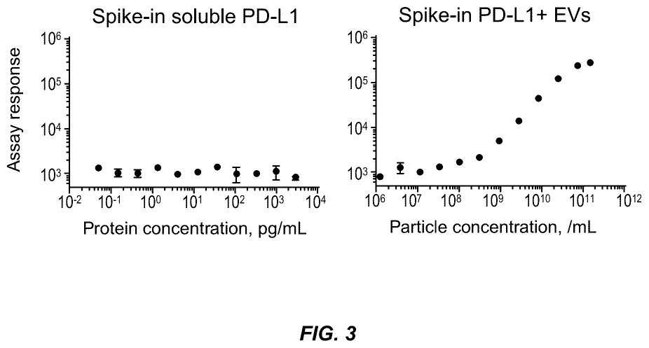

As used throughout, a donor molecule or donor probe refers to a molecule that absorbs energy, and then re-emits at least a portion of the energy over time. Thus, an acceptor molecule or acceptor probe refers to a molecule that will accept energy from a donor molecule, directly or indirectly through another chemical mediator, thus decreasing the donor's emission intensity and excited-state lifetime. Thus, provided that a donor molecule and an acceptor molecule are proximally situated, i.e., physically located sufficiently close to each other, the two molecules function together and, upon excitation with an appropriate wavelength, the donor molecule transfers a precise amount of energy to the acceptor molecule. See, for example, Beaudet et al., “AlphaLISA immunoassays: the no-wash alternative to ELISAs for research and drug discovery,” Nature Methods 5, an8-an9 (2008)). Since the methods provided herein require proximity between the first target protein (for example, PD-L1) and a second target protein (an EV-specific protein) on the same EV, the methods preferably detect EV PD-L1 over soluble forms of PD-L1 in the plasma sample from the subject. See, for example, FIG. 3, showing EV PD-L1 is distinguished from soluble PD-L1 using the methods provided herein. In this example, donor beads contain a photosensitizer, phthalocyanine, which converts ambient oxygen to a high-energy and reactive form of 02, singlet oxygen, upon illumination at 680 nm. Like other excited molecules, singlet oxygen has a limited lifetime prior to falling back to ground state. Within its 4 μsec half-life, singlet oxygen can diffuse approximately 200 nm in solution. If an acceptor bead is within 200 nm, energy is transferred from the singlet oxygen to thioxene derivatives within the acceptor bead, subsequently culminating in light production at 615 nm. In the absence of an acceptor bead, singlet oxygen falls to ground state and no signal is produced. One of skill in the art would understand that the methods are not limited to the use of excitation at 680 nm and light emission at 615 nm, as numerous donor-acceptor pairs and their corresponding excitation/emission wavelengths are known in the art. For example, and not to be limiting, donor-acceptor pairs that use the combination of excitation at 680 nm and light emission at 520-620 nm, or the combination of excitation at 620 nm and light emission at 665 nm can be used.

Any method now known in the art or identified in the future for detecting the interaction between a donor molecule and an acceptor molecule described herein can be used to detect the presence of a target protein, for example, a protein expressed by cancer cells. In some methods, the interaction between the donor molecule and the acceptor molecule is measured using a method selected from the group consisting of AlphaLISA® assay (Beaudet et al.), sandwich

ELISA, homogeneous time-resolved fluorescence (HTRF) (Degorce et al., “HTRF: A Technology Tailored for Drug Discovery; A Review of Theoretical Aspects and Recent Applications,” Curr. Chem. Genomics 3: 22-32 (2009)); Meso Scale Discovery (MSD) platform (Meso Scale Technologies, LLC, Rockville, MD); Quanterix® platform (Quanterix, Billerica, MA), and proximity-dependent ligation/extension assays (Greenwood et al., “Proximity assays for sensitive quantification of proteins,” Biomol. Detect. Quantif. 4: 10-6 (2015)).

In some methods, the plasma sample is contacted with a biotinylated first binding agent that specifically binds the first target protein as well as a streptavidin donor molecule, and a second binding agent that specifically binds to the second target protein, wherein the second binding agent is conjugated to an acceptor molecule. In some methods, the plasma sample is contacted with a biotinylated first binding agent that specifically binds the first target protein, wherein the first binding agent is conjugated to an donor molecule, for example, a streptavidin donor molecule, and a second binding agent that specifically binds to the second target protein, wherein the second binding agent is conjugated to an acceptor molecule.

The first binding agent is optionally selected from the group consisting of an antibody, a protein, a peptide or a chemical. In some methods, the second binding agent is selected from the group consisting of an antibody, a protein, a peptide or a chemical. The terms polypeptide, peptide, and protein, are used interchangeably herein to refer to a polymer of amino acid residues. As used herein, the terms encompass amino acid chains of any length, including full-length proteins, wherein the amino acid residues are linked by covalent peptide bonds.

When the first and/or second binding agent is a protein, the protein can be a full-length protein or a binding portion thereof. In some methods, the protein or binding portion thereof is a natural or cognate ligand for the first target protein or the second target protein.

As used throughout, the term antibody encompasses, but is not limited to, whole immunoglobulin (i.e., an intact antibody) of any class, including polyclonal and monoclonal antibodies. Fragments of antibodies that retain the ability to bind a first or second target protein can also be used in any of the methods taught herein. For example, fragments of anti-PD-1 antibodies that retain the ability to bind to PD-1 and fragments of anti-PD-L1 antibodies that retain the ability to bind to PD-L1 can be used in any of the methods provided herein. Also useful in the methods provided herein are conjugates of antibody fragments and antigen binding proteins (including, for example, single chain antibodies) as described, for example, in U.S. Pat. No. 4,704,692, the contents of which is hereby incorporated by reference in its entirety. Examples of anti-PD-1 antibodies include, but are not limited to, nivolumab (Opdivo®), pembrolizumab (Keytruda®), pidilizumab and BMS-936559. Examples of anti-PDL-1 antibodies include, but are not limited to, atezolizumab (Tecentriq®), durvalumab (Imfinzi®) and avelumab (Bavencio®).

As used herein, cancer is a disease characterized by the rapid and uncontrolled growth of aberrant cells. Cancer cells can spread locally or through the bloodstream and lymphatic system to other parts of the body. The cancer can be a solid tumor. In some methods, the cancer is a blood or hematological cancer, such as a leukemia (e.g., acute leukemia; acute lymphocytic leukemia; acute myelocytic leukemias, such as myeloblastic, promyelocytic, myelomonocytic, monocytic, erythroleukemia leukemias and myelodysplastic syndrome; chronic myelocytic (granulocytic) leukemia; chronic lymphocytic leukemia; hairy cell leukemia), polycythemia vera, or lymphomas (e.g., Hodgkin's disease or non-Hodgkin's disease lymphomas (e.g., diffuse anaplastic lymphoma kinase (ALK) negative, large B-cell lymphoma (DLBCL); diffuse anaplastic lymphoma kinase (ALK) positive, large B-cell lymphoma (DLBCL); anaplastic lymphoma kinase (ALK) positive, ALK+anaplastic large-cell lymphoma (ALCL), acute myeloid lymphoma (AML))), multiple myelomas (e.g., smoldering multiple myeloma, non-secretory myeloma, osteosclerotic myeloma, plasma cell leukemia, solitary plasmacytoma and extramedullary plasmacytoma), Waldenstrom's macroglobulinemia, monoclonal gammopathy of undetermined significance, benign monoclonal gammopathy and heavy chain disease. Solid tumors include, by way of example, bone and connective tissue sarcomas (e.g., bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma), brain tumors (e.g., glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma), breast cancer (e.g., adenocarcinoma, lobular (small cell) carcinoma, intraductal carcinoma, medullary breast cancer, mucinous breast cancer, tubular breast cancer, papillary breast cancer, Paget's disease, and inflammatory breast cancer), adrenal cancer (e.g., pheochromocytoma and adrenocortical carcinoma), thyroid cancer (e.g., papillary or follicular thyroid cancer, medullary thyroid cancer and anaplastic thyroid cancer), pancreatic cancer (e.g., insulinoma, gastrinoma, glucagonoma, vipoma, somatostatin-secreting tumor, and carcinoid or islet cell tumor), pituitary cancers (e.g., Cushing's disease, prolactin-secreting tumor, acromegaly, and diabetes insipidus), eye cancers (e.g., ocular melanoma such as iris melanoma, choroidal melanoma, and ciliary body melanoma, and retinoblastoma), vaginal cancers (e.g., squamous cell carcinoma, adenocarcinoma, and melanoma), vulvar cancer (e.g., squamous cell carcinoma, melanoma, adenocarcinoma, basal cell carcinoma, sarcoma, and Paget's disease), cervical cancers (e.g., squamous cell carcinoma and adenocarcinoma), uterine cancers (e.g., endometrial carcinoma and uterine sarcoma), ovarian cancers (e.g., ovarian epithelial carcinoma, borderline tumor, germ cell tumor, and stromal tumor), esophageal cancers (e.g., squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma), stomach cancers (e.g., adenocarcinoma, fungating (polypoid), ulcerating, superficial spreading, diffusely spreading, malignant lymphoma, liposarcoma, fibrosarcoma, and carcinosarcoma), colon cancers, rectal cancers, liver cancers (e.g., hepatocellular carcinoma and hepatoblastoma), gallbladder cancers (e.g., adenocarcinoma), cholangiocarcinomas (papillary, nodular, and diffuse), lung cancers (e.g., non-small cell lung cancer (NSCLC), squamous cell carcinoma (epidermoid carcinoma), adenocarcinoma, large-cell carcinoma and small-cell lung cancer), testicular cancers (e.g., germinal tumor, seminoma, anaplastic, classic (typical), spermatocytic, nonseminoma, embryonal carcinoma, teratoma carcinoma, choriocarcinoma (yolk-sac tumor)), prostate cancers (e.g., adenocarcinoma, leiomyosarcoma, and rhabdomyosarcoma), penile cancers, oral cancers (e.g., squamous cell carcinoma), basal cancers, salivary gland cancers (e.g., adenocarcinoma, mucoepidermoid carcinoma, and adenoidcystic carcinoma), esopharyngeal cancers (e.g., squamous cell cancer and verrucous cancer), skin cancers (e.g., basal cell carcinoma, squamous cell carcinoma and melanoma, superficial spreading melanoma, nodular melanoma, lentigo malignant melanoma, acral lentiginous melanoma), kidney cancers (e.g., renal cell cancer, adenocarcinoma, hypernephroma, fibrosarcoma, transitional cell cancer (renal pelvis and/or ureter), Wilms' tumor), bladder cancers (e.g., transitional cell carcinoma, squamous cell cancer, adenocarcinoma, and carcinosarcoma). In addition, cancers include myxosarcoma, osteogenic sarcoma, endotheliosarcoma, lymphangio endothelio sarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas. Thus, the cancer can be selected from the group consisting of melanoma, non-small cell lung cancer, small cell lung carcinoma, squamous cell lung cancer, head and neck squamous cell carcinoma, renal cell carcinoma, urothelial carcinoma, breast cancer, Merkel cell carcinoma, hepatocellular carcinoma, esophageal carcinoma, gastric cancer, Hodgkin's lymphoma, cervical cancer, and endometrial cancer.

Optionally, the methods described herein further comprise determining the responsiveness of the subject to an immunotherapy that specifically binds a ligand of the first target protein. In these methods, an increase in the level of the first target protein as compared to a control indicates the subject is nonresponsive to the immunotherapy, whereas a decrease in the level of the target protein as compared to a control indicates the subject is responsive to the immunotherapy. In some methods, a decrease in the level of PD-L1 indicates that the subject is responsive to anti-PD1 immunotherapy. In the methods provided herein, a control level can be, for example, the level of the first target protein in a subject that is nonresponsive to therapy, for example, anti-PD1 therapy, or a reference level. In some methods, the control level of the first target protein on EVs, for example, EV PD-L1 is a threshold level. In some methods, the level of the first target protein, for example, EV PD-L1, in a responder, i.e., a subject responsive to immunotherapy, is at least 30%, 25%, 20%, 15%, 10%, or 5%, below a threshold level. As used herein, the term threshold level or cut-off, is a level of the first target protein that distinguishes between responders and non-responders for a particular therapy.

### Treatment Methods

Optionally, the methods further comprise administering a therapeutically effective amount of an immunotherapy to the subject that is responsive to the immunotherapy, to treat the cancer in the subject. In some methods, the immunotherapy is anti-PD-1 therapy or an anti PD-L1 therapy, wherein an increase in the level of EV PD-L1 as compared to the control indicates the subject is nonresponsive to the anti-PD-1 therapy or the anti-PD-L1 therapy and wherein a decrease in the level of EV PD-L1 as compared to a control indicates the subject is responsive to the anti-PD-1 therapy or the anti-PD-L1 therapy. In some methods, an anti-PD-1 therapy or an anti-PD-L1 therapy to the subject that is responsive to the anti-PD-1 therapy or the anti-PD-L1 therapy.

In some methods, the anti-PD-1 therapy is an inhibitor that blocks or disrupts the interaction between PD-1 and PD-L1, for example, an anti-PD-1 antibody or an anti-PD-L1 antibody. In some methods, the anti-PD-1 antibody is selected from the group consisting of nivolumab (Opdivo®), pembrolizumab (Keytruda®), pidilizumab and BMS-936559. In some methods the anti-PDL-1 antibody is selected from the group consisting of atezolizumab (Tecentriq®), durvalumab (Imfinzi®) and avelumab (Bavencio®). Other PD-L1 inhibitors include, but are not limited to KNO35, CK-301, AUNP12, CA-170 and BMC-986189.

Any of the treatment methods described herein can further comprise administering an effective amount of a second therapeutic agent to the subject. The second therapeutic agent can be selected from the group consisting of a chemotherapeutic agent, an adjuvant, an immunomodulatory agent, a vaccine, a potentiating agent, a tumor antigen, or a combination thereof. It is understood that combinations, for example, a composition comprising immunotherapy and a chemotherapeutic agent, can be administered either concomitantly (e.g., as an admixture), separately but simultaneously (e.g., via separate intravenous lines into the same subject), or sequentially (e.g., one of the compounds or agents is given first followed by the second). Any of the methods provided herein can further comprise radiation therapy or surgery.

Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof.

Also provided herein is a method of determining a subjects responsivity to an immunotherapy or combination of immunotherapy with a second therapeutic agent in a subject by first administering to the subject at least once a therapeutically effective amount of an immunotherapy and/or second therapeutic agent. Before and subsequent to the one or more administrations of the immunotherapy and/or second therapeutic agent, the level of a first target protein on the surface of EVs comprising (a) contacting a plasma sample from the subject with (i) a first binding agent that specifically binds to the first target protein expressed on the surface of EVs, wherein the first binding agent is conjugated to a first member of a proximity pair; and (ii) a second binding agent that specifically binds to a second target protein, wherein the second target protein is selectively expressed on the surface of EVs and wherein the second binding agent is conjugated to a second member of a proximity pair; and (b) detecting the level of the first target protein expressed on the surface of the EVs by detecting proximity of the first member of the proximity pair and the second member of the proximity pair, wherein the proximity occurs upon binding of the first binding agent to the first target protein and binding of the second binding agent to the second target protein on the same EV.

A decrease in the level of the first target protein as compared to a pretreatment control (the level before treatment) indicates the subject is not responding to the immunotherapy, and an increase in the level of the first target protein as compared to a pretreatment control indicates the subject is responding to the immunotherapy. Upon determining the subject is not responsive, additional steps can be taken, such as increasing the doses of the immunotherapy and/or second therapeutic agent or by administering a different immunotherapy and/or second therapeutic agent.

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult, newborn and pediatric subjects, whether male or female, are intended to be covered. As used herein, patient or subject may be used interchangeably and can refer to a subject with or at risk of developing a disorder, or relapsing. The term patient or subject includes human and veterinary subjects. In any of the methods provided herein, the subject can be a subject diagnosed with cancer, an infection or an autoimmune disease.

As used herein the terms treatment, treat, or treating refers to a method of reducing one or more of the effects of the disorder or one or more symptoms of the disorder, for example, cancer in the subject. Thus in the disclosed methods, treatment can refer to a 10%, 20%, 30%, 40%, 50%, 60%, 70%, 80%, 90%, or 100% reduction in the severity of cancer. For example, a method for treating cancer is considered to be a treatment if there is a 10% reduction in one or more symptoms of the cancer in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disorder.

As used herein, the term therapeutically effective amount or effective amount refers to an amount of a chemotherapeutic agent, immunotherapeutic agent, etc. described herein, that, when administered to a subject, is effective, alone or in combination with additional agents, to treat a disease or disorder either by one dose or over the course of multiple doses. A suitable dose can depend on a variety of factors including the particular cells or agent used and whether it is used concomitantly with other therapeutic agents. Other factors affecting the dose administered to the subject include, e.g., the type or severity of the disease. For example, a subject having NSCLC may require administration of a different dosage of an immunotherapeutic agent and/or a chemotherapeutic agent than a subject with melanoma.

The effective amount of an immunotherapeutic agent can be determined by one of ordinary skill in the art and includes exemplary dosage amounts for a mammal of from about 0.5 to about 200 mg/kg of body weight of active compound per day, which can be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, about 5 mg/kg of body weight, about 3 mg/kg of body weight, or about 2.5 mg/kg of body weight of active compound per day. Other factors that influence dosage can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

As used herein, administering or administration refers to the act of introducing, injecting or otherwise physically delivering a substance as it exists outside the body (e.g. an immunotherapeutic agent) into a subject, such as by mucosal, intradermal, intravenous, intratumoral, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof.

Any of the therapeutic agents described herein (for example, a chemotherapeutic agent, a vaccine, an immunotherapeutic, etc.) are administered in a number of ways depending on whether local or systemic treatment is desired, and on the area to be treated. The compositions are administered via any of several routes of administration, including orally, parenterally, intramucosally, intravenously, intraperitoneally, intraventricularly, intramuscularly, subcutaneously, intracavity or transdermally. Administration can be achieved by, e.g., topical administration, local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; and European Patent Nos. EP488401 and EP 430539.

In some methods, a therapeutic agent can be delivered to the subject by way of an implantable device based on, e.g., diffusive, erodible, or convective systems, osmotic pumps, biodegradable implants, electrodiffusion systems, electroosmosis systems, vapor pressure pumps, electrolytic pumps, effervescent pumps, piezoelectric pumps, erosion-based systems, or electromechanical systems. Effective doses for any of the administration methods described herein can be extrapolated from dose-response curves derived from in vitro or animal model test systems.

Any of the therapeutic agents described herein, for example, immunotherapeutic agents or chemotherapeutic agents, can be formulated as a pharmaceutical composition. In some embodiments, the pharmaceutical composition can further comprise a carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Depending on the intended mode of administration, a pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

The use herein of the terms including, comprising, or having and variations thereof, is meant to encompass the elements listed thereafter and equivalents thereof as well as additional elements. Embodiments recited as including, comprising, or having certain elements are also contemplated as consisting essentially of and consisting of those certain elements. As used herein, and/or refers to and encompasses any and all possible combinations of one or more of the associated listed items, as well as the lack of combinations where interpreted in the alternative (or).

As used herein, the transitional phrase consisting essentially of (and grammatical variants) is to be interpreted as encompassing the recited materials or steps and those that do not materially affect the basic and novel characteristic(s)″ of the claimed invention. See, In re Herz, 537 F.2d 549, 551-52, 190 U.S.P.Q. 461, 463 (CCPA 1976) (emphasis in the original); see also MPEP § 2111.03. Thus, the term, consisting essentially of, as used herein, should not be interpreted as equivalent to “comprising.”

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Blood Plasma Preparation

Patient plasma samples were centrifuged at 3000×g for 10 min at 4° C. and supernatants were collected. Clarified plasma samples were then diluted with 1× AlphaLISA® buffer (Perkin Elmer, Waltham, MA) (25 mM Hepes, 0.1% (w/v) casein, 10 mM sodium phosphate, 15 mM NaCl, 1 mg/mL dextran 500, 0.5% (v/v) Tween-20, 0.05% (v/v) proclin-300, pH 7.4) in a 1:1 volume ratio; or with 2× enhanced blocking AlphaLISA buffer (50 mM Hepes pH 7.4, 0.2% (w/v) casein, 20 mM sodium phosphate, 30 mM NaCl, 2 mg/mL dextran 500, 1% (v/v) Tween-20, 0.1% (v/v) proclin-300, 40% (v/v) Pierce Protein-Free (PBS) Blocking Buffer (Thermo Fisher, Waltham, MA)).

### Bead Conjugation

Anti-CD9 antibody (clone HI9a, manufactured by BioLegend (San Diego, CA)) was conjugated to unconjugated acceptor beads using a 10:1 challenge ratio of beads to antibody by mass, following the manufacturer suggested protocol. In brief, the anti-CD9 antibody was buffer exchanged into PBS and concentrated to 1 mg/mL. Next, 0.1 mg of antibody was combined with 1 mg of washed and pelleted acceptor beads, 1.25 μL of 10% Tween-20, 10 μL of 400 mM NaBH₃CN, and 88.75 μL of 100 mM Hepes pH 7.4, for a total reaction volume of 200 μL. The mixture was then incubated for 20 hours at 37° C. with mild agitation. The conjugated beads were then blocked by adding 10 μL of 65 mg/mL carboxymethoxylamine and incubated at 37° C. for 1 hour with gentle shaking. Last, the beads were washed and pelleted twice before resuspending the beads with 200 μL of PBS+0.05% Proclin-300 for a final bead concentration of 5 mg/mL.

Recombinant PD-1 appended with the C-terminal lysine rich tripeptide SKK was conjugated to unconjugated donor beads using a 10:1 challenge ratio of beads to protein by mass, following the manufacturer suggested protocol as above.

### Recombinant Mutant PD-1 Generation

To effect high affinity bead binding to vesicular PD-L1, a high affinity, mutant human PD-1 ectodomain constructs, identified via phage display (Li et al., “High-affinity PD-1 molecules deliver improved interaction with PD-L1 and PD-L2,” Cancer Sci. 109(8): 2435-2445 (2018)), were used. In one iteration, nucleic acids encoding an N-terminal mammalian membrane metallopeptidase secretion signal, a C-terminal Avitag and decahistidine tags were appended to a nulceic acid encoding a recombinant PD-1 protein, and the construct was subcloned into a mammalian expression vector for protein generation. Additionally, a Cys93->Ser mutation was introduced to eliminate protein dimerization after purification. In another iteration, a C-terminal decahistidine tag and a lysine rich tripeptide (SKK) were appended to a nucleic acid encoding a recombinant PD-1 protein, and the construct was subcloned into a mammalian expression vector for protein generation. The final constructs were transfected into Expi293F cells (Thermo Fisher (Waltham, MA)) for secreted expression and purification via immobilized metal affinity chromatography on an AKTA fast performance liquid chromatography instrument, eluting with 250 mM imidazole. The elution fractions were collected and loaded onto the size exclusion chromatography column for further purification and buffer exchange.

The Avitag purified protein was then site-specifically biotinylated using E. coli biotin ligase (BirA500 kit, Avidity Biosciences (La Jolla, CA), using modified protocol based on the manufacturer's manual. After incubation for 5 hrs at room temperature, the reaction mixture was captured on nickel-immobilized resin and washed with 25 mM imidazole to remove excess biotin and biotin ligase before eluting with 250 mM imidazole in phosphate buffered saline. The protein was then desalted using 7 kDA molecular weight cut-off Zeba columns (Thermo Fisher) and exchanged into phosphate buffered saline. The purity was assessed using SDS-PAGE before aliquoted and stored at −80° C.

### EV PD-L1 Detection

For detection of PD-L1 on EVs in plasma samples using biotinylated PD-1, 8.33 μL of biotinylated recombinant PD-1 protein (107 ng/mL) was added to each well of a 96-well light gray half-area AlphaPlate (Perkin Elmer, cat #6052340). Then 50 μL diluted plasma sample was added into the plate in triplicate. After incubation in the dark for 2 hrs (23° C., 500 rpm), 8.33 μL mix of streptavidin donor beads (160 μg/mL, Perkin Elmer, cat #6760002S), and anti-CD9 acceptor beads (20 μg/mL) was added to each well and the plate was incubated in the dark for another 2 hrs (23° C., 500 rpm). The plate was read at 615 nm on a BMG CLARIOstar plate reader with an AlphaScreen installed module using 9 mm focal height and 3500 gain.

When beads directly conjugated to PD-1 were used for detection of PD-L1 on EVs in plasma samples, a 50 μL diluted plasma sample was added into the plate in triplicate. PD-1 conjugated donor beads were mixed with anti-CD9 conjugated acceptor beads at a final concentration of 80 μg/mL and 10 μg/mL, respectively in AlphaLISA buffer. Then, 16.67 of the bead mixture was added to each sample. After incubation in the dark for 2 hrs (23° C., 500 rpm), the plate was read as above.

### Results

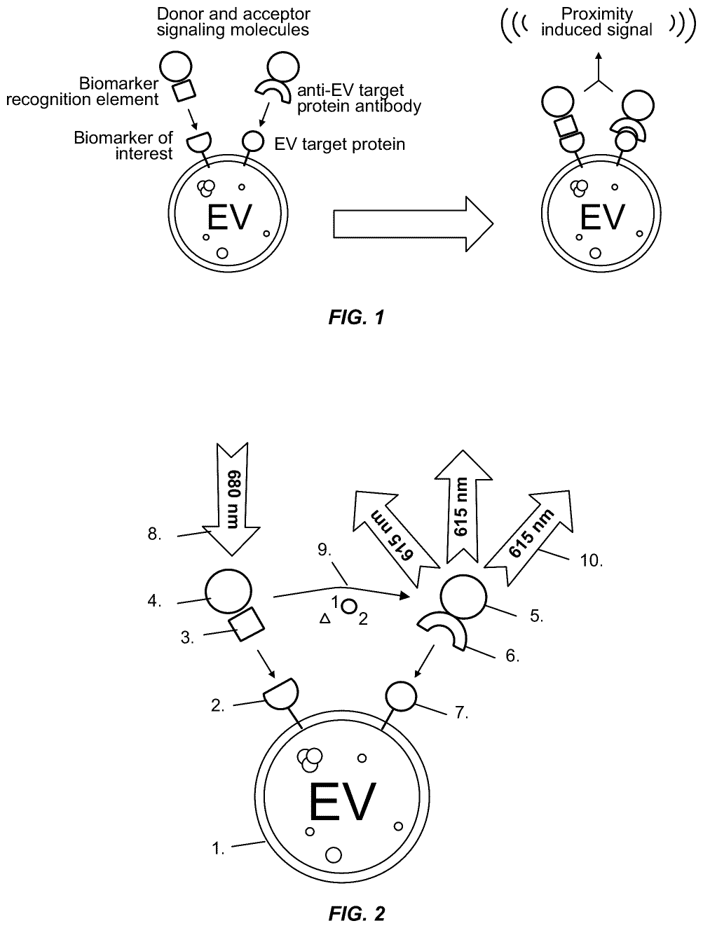

As shown in FIG. 1, using the methods described herein, a signal based on the coexpression of a biomarker of interest (for example, PD-L1) and a EV-specific protein on the surface of blood plasma EVs was generated. This enables the analysis of vesicle-specific biomarker expression in plasma and is amenable to several assay formats including AlphaLISA®, sandwich ELISA, homogeneous time-resolved fluorescence (HTRF), Meso Scale Discovery (MSD) platform, Quanterix platform, and proximity-dependent ligation/extension assays.

In a specific example, as shown in FIG. 2, EVs (1) expressing a biomarker of interest ((2) PD-L1 protein), were bound to a biomarker recognition element ((3) high-affinity PD-1 molecule) that was linked to an AlphaLISA Acceptor Bead (4). An AlphaLISA® Donor Bead (5) linked to a vesicle-specific antibody (6) bound to its cognate vesicle specific surface protein ((7) CD9 protein). Excitation by 680 nm light (8) caused the Donor Bead to emit single oxygen molecules (9) that travelled in solution to activate the Acceptor Bead which then emitted a sharp peak of light at 615 nm (10). This light emission was detected by an Alpha-enabled reader.

As shown in FIG. 3, the EV PD-L1 assay detected vesicle-bound, but not free/soluble PD-L1. The assay does not register signal above background when increasing titrations of soluble PD-L1 are added to the assay (FIG. 3, left panel). Cell line-derived EVs co-expressing surface CD9 and PD-L1 spiked into an EV PD-L1 assay elicit signal in a vesicle/particle concentration dependent manner (FIG. 3, right panel).

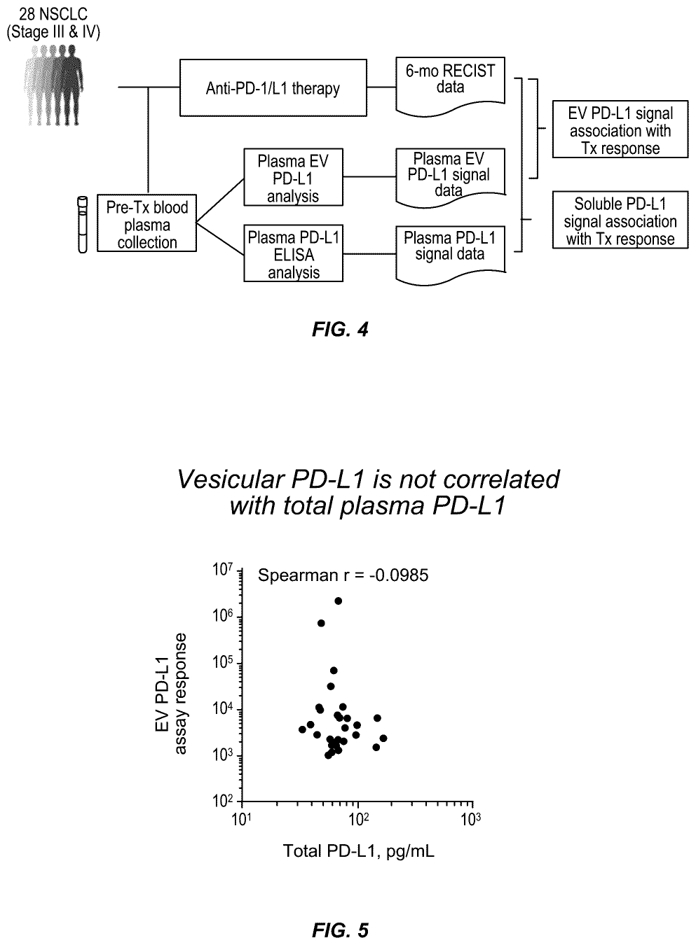

To assess the diagnostic use of the EV PD-L1 assay, EV PD-L1 and total PD-L1 (using a commercial ELISA kit) from the blood of non-small cell lung cancer patients prior to anti-PD-1/L1 therapy, were evaluated. FIG. 4 shows the study design. Pre-treatment EV and total PD-L1 data was integrated with 6 month treatment response data to determine associations with treatment outcomes.

As shown in FIG. 5, EV PD-L1 (y-axis) and total PD-L1 levels (x-axis) in pretreatment plasma samples are not correlated (Spearman correlation, r). These data further support the difference in specificity between EV PD-L1 and commercial ELISA PD-L1 assays.

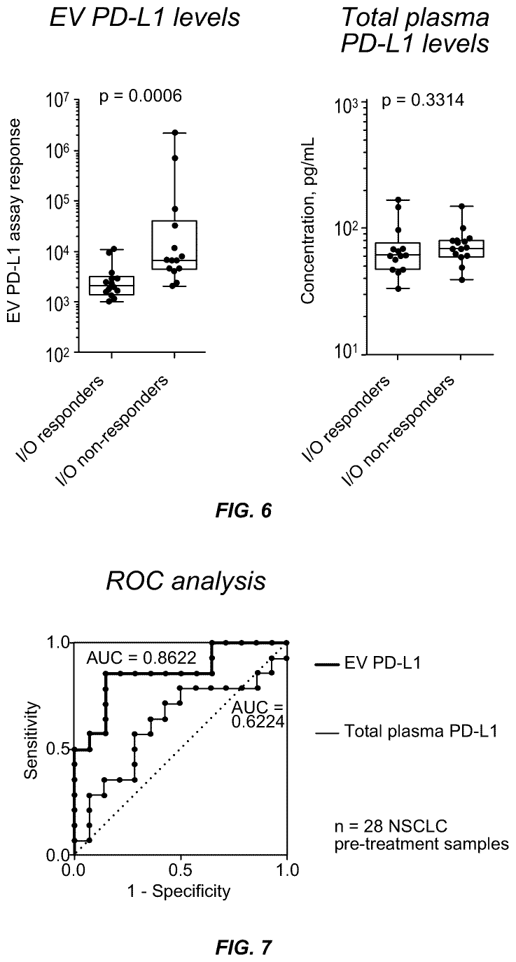

FIG. 6 shows that EV PD-L1 levels are significantly different (t-test, p-value<0.001) in pretreatment plasma from patients that exhibit tumor shrinkage or stable disease (I/O responders), as compared to patients whose tumors continue to grow during treatment (I/O non-responders) based on a 6 month RECIST data (left panel). However no significant difference in total PD-L1 was observed in I/O-responders and I/O non-responders pretreatment plasma samples. These data suggest elevated EV PD-L1 levels before therapy is associated with treatment response outcomes (right panel).

A receiver operating characteristic (ROC) curve compares the accuracy of EV PD-L1 and total plasma PD-L1 assays in discriminating I/O-responder and I/O non-responder patients via analysis of pretreatment plasma samples. Area under the curve (AUC) calculations reveal the superior accuracy of EV PD-L1 vs total plasma PD-L1 is classifying treatment response outcomes (FIG. 7).

1. A method for detecting in a subject with cancer the level of a first target protein expressed on the surface of extracellular vesicles (EVs), comprising:
a) contacting a plasma sample from the subject with
(i) a first binding agent that specifically binds to the first target protein expressed on the surface of EVs, wherein the first binding agent is conjugated to a first member of a proximity pair; and
(ii) a second binding agent that specifically binds to a second target protein, wherein the second target protein is selectively expressed on the surface of EVs and wherein the second binding agent is conjugated to a second member of a proximity pair; and
b) detecting the level of the first target protein expressed on the surface of the EVs by detecting proximity of the first member of the proximity pair and the second member of the proximity pair, wherein the proximity occurs upon binding of the first binding agent to the first target protein and binding of the second binding agent to the second target protein on the same EV.

2. The method of claim 1, wherein the first member of the proximity pair is an acceptor molecule and the second member of the proximity pair is a donor molecule.

3. The method of claim 1, wherein the first member of the proximity pair is a donor molecule and the second member of the proximity pair is an acceptor molecule.

4. The method of claim 1, wherein the second target protein is selected from the group consisting of CD9, CD63 and CD81.

5. The method of claim 1, wherein the first target protein is selected from the group consisting of programmed cell death protein 1 (PD-1), programmed cell death protein ligand 1 (PD-L1), programmed cell death protein ligand 2 (PD-L2), cytotoxic T-lymphocyte-associated protein 4 (CTLA4), Lymphocyte-Activation Gene 2 (LAG2), transforming growth factor β1 (TGF-β1), NKG2D, T-cell immunoglobulin and mucin domain-3 (TIM-3), Lymphocyte-Activation Gene 3 (LAG-3), and V-domain Ig suppressor of T cell activation (VISTA).

6. The method of claim 2, wherein the first binding agent is conjugated to the first member of the donor-acceptor pair and/or the second binding agent is conjugated to the second member of the donor-acceptor pair via a biotin-streptavidin or covalent linkage.

7. The method of claim 6, wherein the method comprises contacting the plasma sample with a biotinylated first binding agent that specifically binds the first target protein, a streptavidin donor molecule, and a second binding agent that specifically binds to the second target protein, wherein the first binding agent is conjugated to an acceptor molecule.

8. The method of claim 1, wherein the proximity between the donor molecule and the acceptor molecule is measured by AlphaLISA® assay.

9. The method of claim 1, wherein the first target protein is PD-L1.

10. The method of claim 9, wherein the second target protein is selected from the group consisting of CD9, CD63 and CD81.

11. The method of claim 1, wherein the method does not comprise isolating extracellular vesicles from the plasma sample.

12. The method of claim 1, further comprising determining the responsiveness of the subject to an immunotherapy that specifically binds a ligand of the first target protein, wherein an increase in the level of the first target protein as compared to a control indicates the subject is nonresponsive to the immunotherapy, and a decrease in the level of the target protein as compared to a control indicates the subject is responsive to the immunotherapy.

13. The method of claim 12, further comprising administering the immunotherapy to the subject that is responsive to the immunotherapy.

14. The method of claim 12, wherein the immunotherapy is an anti-PD-1 therapy or an anti PD-L1 therapy, wherein an increase in the level of EV PD-L1 as compared to the control indicates the subject is nonresponsive to the anti-PD-1 therapy or the anti-PD-L1 therapy, and wherein a decrease in the level of EV PD-L1 as compared to a control indicates the subject is responsive to the anti-PD-1 therapy or the anti-PD-L1 therapy.

15. The method of claim 14, wherein the control level of EV PD-L1 is a threshold level.

16. The method of claim 14 or 15, wherein the method further comprises administering an anti-PD-1 therapy or an anti-PD-L1 therapy to the subject that is responsive to the anti-PD-1 therapy or the anti-PD-L1 therapy.

17. The method of claim 16, wherein the anti-PD-1 therapy is a PD-1 or a PD-L1 inhibitor.

18. The method of claim 17, wherein the inhibitor is an antibody.

19. The method of claim 18, wherein the antibody is selected from the group consisting of pembrolizumab, nivolumab, atezolizumab, avelumab and durvalumab.

20. The method of claim 1, wherein the cancer is selected from the group consisting of melanoma, non-small cell lung cancer, small cell lung carcinoma, squamous cell lung cancer, head and neck squamous cell carcinoma, renal cell carcinoma, urothelial carcinoma, breast cancer, Merkel cell carcinoma, hepatocellular carcinoma, esophageal carcinoma, gastric cancer, Hodgkin's lymphoma, cervical cancer, and endometrial cancer.
